(19) United States
(12) Patent Application Publication
(10) Pub. No.: US 2007/0255414 A1
(43) Pub. Date: Nov. 1, 2007

(54) Intervertebral implants with one or more covers and methods of use

(75) Inventors: Anthony J. Melkent, Memphis, TN (US); Lindsey Gardner Waugh, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/414,888

(22) Filed: May 1, 2006

(51) Int. Cl. A61F 2/44

(52) U.S. Cl. 623/17.16; 623/17.11

(57) ABSTRACT

The present application is directed to intervertebral implants. The implants may include a body with superior and inferior surfaces, and a sidewall. An interior section may be positioned within the body and sized to contain bone growth material. An opening extends through one or both of the superior and inferior surfaces to access the interior section. A cover may be attached to the body to extend across the opening. In some embodiments, the cover includes teeth that engage a vertebral member. The cover may further include a plate that extends across the opening, and an attachment mechanism to attach the cover to the body. One method of using the implant may include inserting bone growth material into the interior section and attaching the cover to close the opening. The implant may then be inserted into the intervertebral space.

### BACKGROUND

The present application is directed to an intervertebral implant and, more specifically, to an intervertebral implant with one or both of the inferior and superior surfaces including an opening with a cover to access an interior section.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion.

Various procedures include replacing the entirety or a section of a vertebral member, the entirety or a section of an intervertebral disc, or both. One or more replacement implants may be inserted to replace the damaged vertebral members and/or discs. The implants may further include bone growth material to facilitate fusion of the implant to one or both adjacent vertebral members. The implant should provide for housing the bone growth material, and prevent inadvertent removal of the material from the implant.

### SUMMARY

The present application is directed to intervertebral implants. The implants may include a body with superior and inferior surfaces, and a sidewall. An interior section may be positioned within the body and sized to contain bone growth material. An opening extends through one or both of the superior and inferior surfaces to access the interior section. A cover may be attached to the body to extend across the opening. In some embodiments, the cover includes teeth that engage a vertebral member. The cover may further include a plate that extends across the opening, and an attachment mechanism to attach the cover to the body.

### BRIEF DESCRIPTION OF THE DRAWINGS

FIG. 1 is a side view illustrating an implant positioned within an intervertebral space between vertebral members according to one embodiment.

FIG. 2A is a perspective view illustrating an implant with a cover removed from a body according to one embodiment.

FIG. 2B is a perspective view illustrating an implant with a cover attached to a body according to one embodiment.

FIG. 3A is a cross section view cut along line 3A-3A of FIG. 2A illustrating an implant according to one embodiment.

FIG. 3B is a partial cross section view illustrating teeth according to one embodiment.

FIG. 4 is a perspective view illustrating an implant with a cover attached to a body according to one embodiment.

FIG. 5 is a cross section view cut along line 5A-5A of FIG. 2A illustrating a cover according to one embodiment.

FIG. 6 is a perspective view illustrating a cover according to one embodiment.

FIG. 7 is a top view illustrating an implant with a cover removed from a body according to one embodiment.

FIG. 8 is a top view illustrating an implant with a cover attached to a body according to one embodiment.

FIG. 9 is a top view illustrating a cover according to one embodiment.

FIG. 10 is a top view illustrating a cover according to one embodiment.

FIG. 11 is a top view illustrating a cover according to one embodiment.

FIG. 12 is a perspective view illustrating an implant with a cover removed from a body according to one embodiment.

FIG. 13 is a perspective view of an implant with a pair of covers removed from a body according to one embodiment.

FIG. 14 is a cross section view of an implant according to one embodiment.

FIGS. 15A and 15B are top views illustrating an implant with a cover attached to a body at different angular positions according to one embodiment.

FIG. 16 is a top view illustrating an implant with a cover attached to a body according to one embodiment.

FIG. 17 is a top view illustrating an implant with a cover attached to a body according to one embodiment.

### DETAILED DESCRIPTION

The present application is directed to intervertebral implants. The implants include a body that includes inferior and superior surfaces that face the vertebral members. A sidewall extends between the inferior and superior faces. An interior section is formed within body and is sized to hold bone growth material. One or both of the inferior and superior faces include enlarged openings to access the interior section. Each opening further includes a removable cover. The cover can be removed to pack bone growth material into the interior section, and reattached to prevent the inadvertent escape of the bone growth material. The cover may also include teeth that bite against the vertebral member and prevent expulsion.

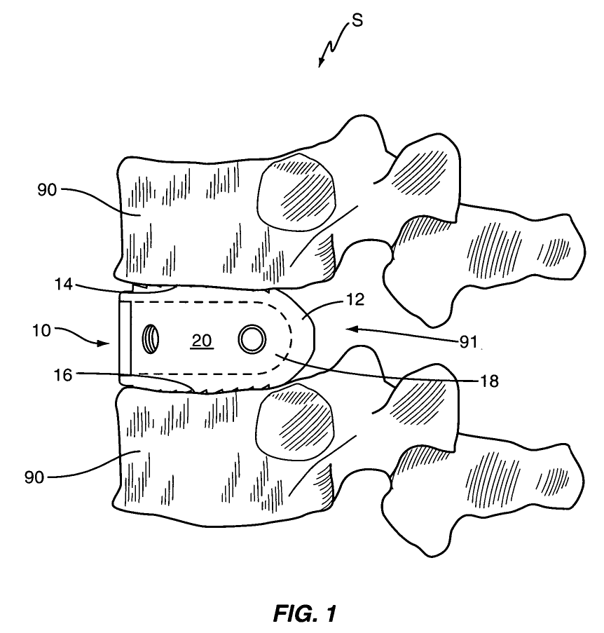

FIG. 1 illustrates a lateral view of one embodiment of an implant 10 positioned within a patient's spine S. The implant 10 comprises a body 12 sized to fit within the intervertebral space 91 between adjacent vertebral members 90. The body 12 includes superior surface 14, an inferior surface 16, and a surrounding sidewall 18 that substantially enclose an interior section 20. The superior and inferior surfaces 14, 16 may be substantially flat, or may include a convex shape to conform to the shape of the vertebral members 90. In FIG. 1, the superior surface 14 contacts the lower surface of the superior vertebral member 90, while the inferior surface 16 contacts the upper surface the inferior vertebral member 90. The interior section 20 receives bone growth material that, as described below in more detail, grows through the superior and inferior surfaces 14, 16 to fuse with the vertebral members 90.

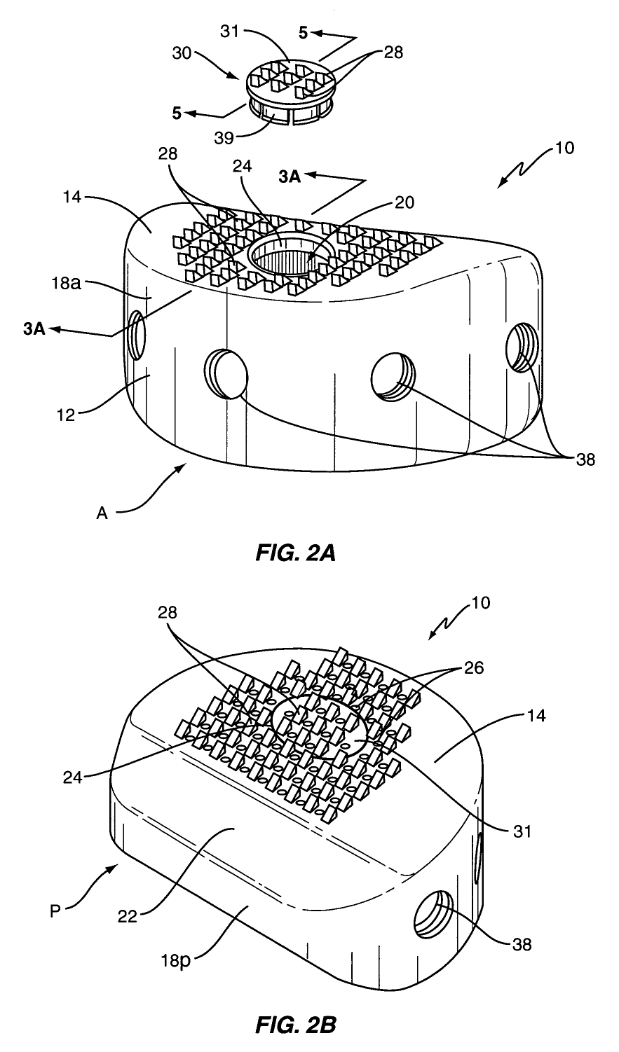

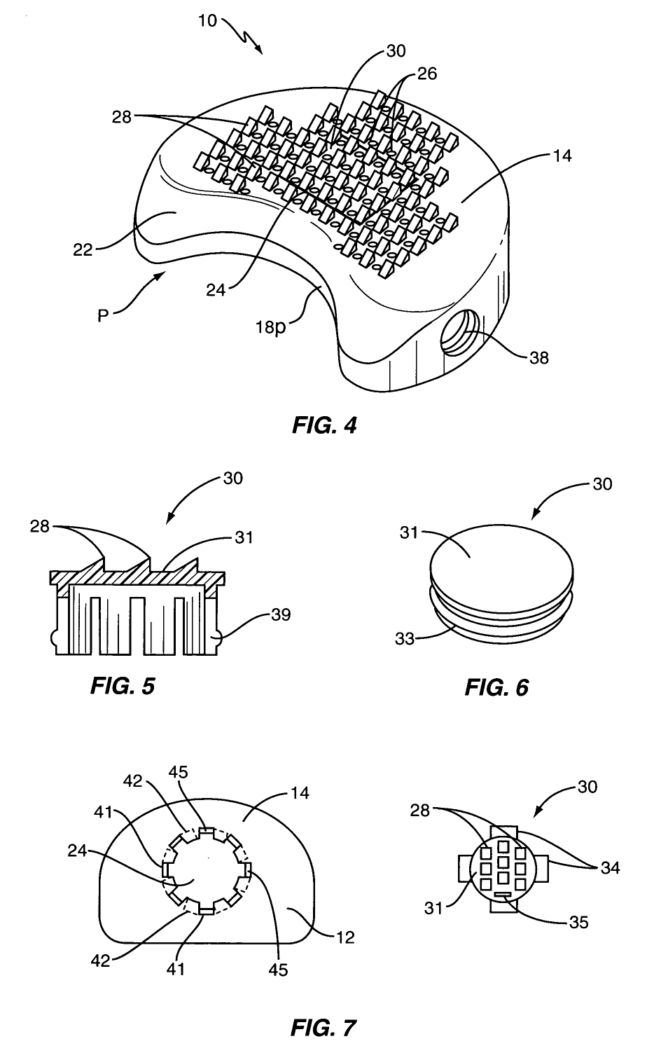

FIGS. 2A and 2B illustrate perspective views of an implant 10 formed according to one embodiment. The body 12 includes an anterior side A (FIG. 2A), and a posterior side P (FIG. 2B). The sidewall 18a on the anterior side A may be arcuate to conform to the shape of the anterior portion of the vertebral members 90. The sidewall 18p on the posterior side P of body 12 is formed to comprise a substantially straight edge. Sidewall 18p may also include a curved shape as illustrated in FIG. 4.

One or both of the superior and inferior surfaces 14, 16 may include a tapered surface 22 that extends downward on the posterior side P to meet the peripheral edges of sidewall 18p. The tapered surface 22 reduces the height of the posterior side P of body 12, relative to the anterior side A, to facilitate surgical insertion during an anterior approach.

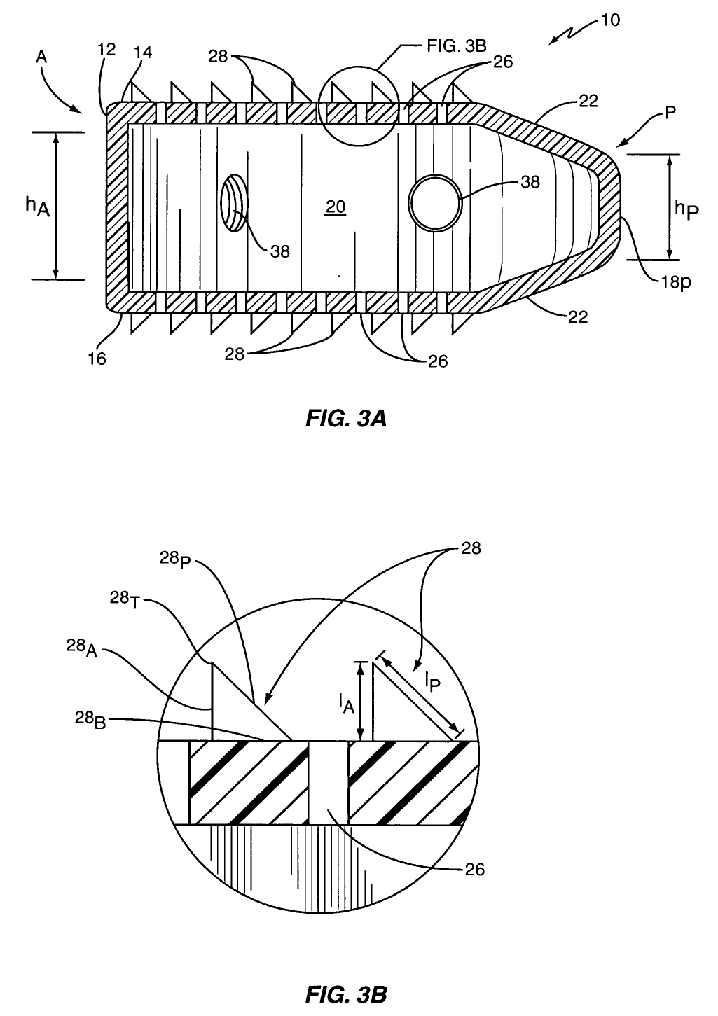

As seen in FIG. 3A, a cross-sectional view of this implant 10 illustrates the body 12 including a substantially bullet-shaped lateral profile. Particularly, the tapered surfaces 22 slope to meet sidewall 18_{P}. The tapered surfaces 22 may slope at any desired angle; however in one embodiment, the tapered surfaces 22 slope at substantially the same angle. The tapered surfaces 22 result in a height h_{P} of the sidewall 18_{P} being less than a height h_{A} of the opening 24. The reduced height of the body 12 on the posterior side P facilitates insertion of the implant between the vertebral members 90. The reduced height of the sidewall 18_{P} and the tapered surfaces 22 separate the vertebral members 90 and facilitate entry of the implant 10 into the intervertebral space 91. FIG. 3A illustrates the sidewall 18_{P} being substantially flat. In another embodiment, the tapered surfaces 22 substantially meet to form a radius transition with the height h_{P} being less than that of FIG. 3A.

One or more receptacles 38 may be formed in the sidewall 18 to receive an insertion tool to place the implant 10 within the patient. The receptacles 38 may be positioned at various locations around the sidewall 18. In one embodiment, receptacles 38 are threaded to engage with a threaded insertion tool. Various other attachment options may also be utilized for attaching the implant 10 to the insertion tool.

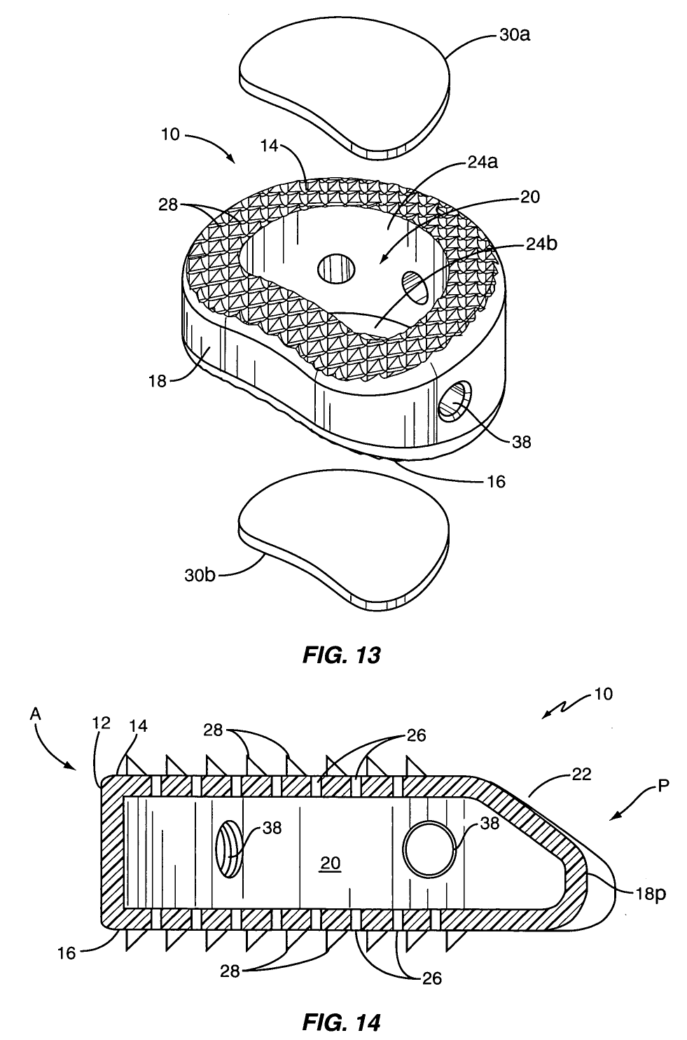

A plurality of small openings 26 may be machined into the superior and inferior surfaces 14, 16. The openings 26 open into the interior section 20 to allow the bone growth material to grow through the surfaces 14,16 and fuse with the vertebral members 90. The openings 26 may be distributed across the surfaces 14, 16 in a variety of patterns. For example, the openings 26 may be arranged in a radial pattern, or a staggered pattern, across one or both of the superior and inferior surfaces 14,16. Cover 30 may also include a grate configuration with a series of small apertures 26 as illustrated in FIGS. 9,10, and 11. These apertures 26 form a pattern that provides for fusion with the vertebral members 90. In some embodiments, the cover 30 is substantially smooth as illustrated in FIGS. 6 and 13.

In another embodiment, one or both of the superior and inferior surfaces 14, 16 are constructed of a porous mesh material extending between the sidewall 18. In other embodiments, only portions of one or both of the superior and inferior surfaces 14, 16 are constructed of a porous mesh material.

Teeth 28 may be distributed across one or both of the superior and inferior surfaces 14, 16. FIGS. 3A-3B illustrate the teeth 28 as they might be formed according to one embodiment. In this embodiment, the teeth 28 form a serrated pattern on each surface 14, 16. Each tooth 28 comprises a polygon with a base 28_{B} and an opposing tip 28_{T}. The teeth 28 may be solid or hollow, but the surface area of the base 28_{B} is larger than the surface area of the tip 28_{T}. Each tooth 28 is further formed such that a length I_{A} of a first side 28_{A} of tooth 28 is shorter than a length I_{P} of a second side 28_{P} of tooth 28. Thus formed, the teeth 28 are oriented to slant slightly towards one side of the body 12. This facilitates the insertion of the implant 10 into the intervertebral space using a predetermined approach. In one embodiment, the first side 28_{A} faces in an anterior direction and the second side 28_{P} faces in a posterior direction to facilitate insertion from an anterior approach. The teeth 28 may also be oriented in other directions to facilitate other approaches.

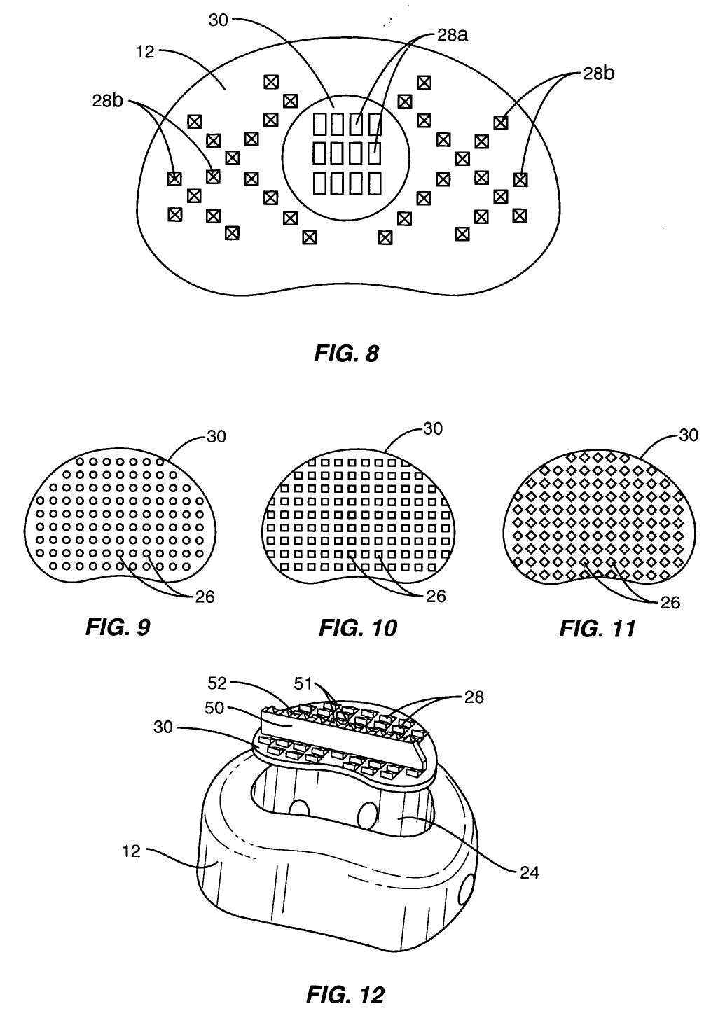

Once the implant 10 is inserted, the tips 28_{T} of the teeth 28 grip the surfaces of the vertebral members 90. In this position, the teeth 28 resist removal of the implant 10 in the direction of insertion and maintain the alignment of implant 10 within the intervertebral space 91. In other embodiments, teeth 28 include other shapes and sizes. FIG. 8 illustrates an embodiment with symmetrical teeth 28b with the sides including the same length. Examples of symmetrical teeth 28b include pyramid-shaped or conical teeth each with equal sides. In another embodiment, one or both surfaces 14, 16 include teeth with two or more different shapes and sizes.

In the embodiment of FIGS. 2A and 2B, an opening 24 is formed in the superior surface 14 that opens into the interior section 20. The opening 24 is larger than the apertures 26 and provides access to the interior section 20 to position bone growth material that will later fuse with the adjacent vertebral members 90. The interior section 20 may be formed as a single cavity within an interior of the body 12, or may comprises a plurality of intercommunicating cavities that may be at least partially separated by one or more inner walls (not shown). The opening 24 may include a variety of shapes and sizes including substantially circular as illustrated in FIG. 2A, rectangular as illustrated in FIG. 4, and substantially D-shaped as illustrated in FIG. 13.

A cover 30 is removably attached to the body 12 to extend over the opening 24. In the embodiment illustrated in FIG. 2A, cover 30 includes a plate 31 sized to extend across the opening 24. The plate 31 may include the same or different thickness as the surfaces 14, 16. The plate 31 may be substantially flat, or may be contoured to match the shape of the surfaces 14, 16. Teeth 28 may extend outward from the plate 31 and are sized to engage the vertebral member 90, and may include a variety of shapes and sizes. Attachment mechanism 32 may extend outward from the underside of the plate 31 to attach to the body 12. The plate 31 may not include teeth 28 as illustrated in the embodiment of FIG. 13.

FIG. 2B illustrates the cover 30 attached to the body 12 and extending across the opening 24. In this embodiment, the plate 31 is substantially the same shape and size of the opening 24. In other embodiments, the plate 31 may include a different shape and/or size resulting in openings formed between the edges of the opening 24 and the plate 31. In the embodiment of FIG. 2B, the surface of the plate 31 is substantially flush with the surface of the superior surface 14. In other embodiments, the plate surface may extend above or be recessed relative to the superior surface 14.

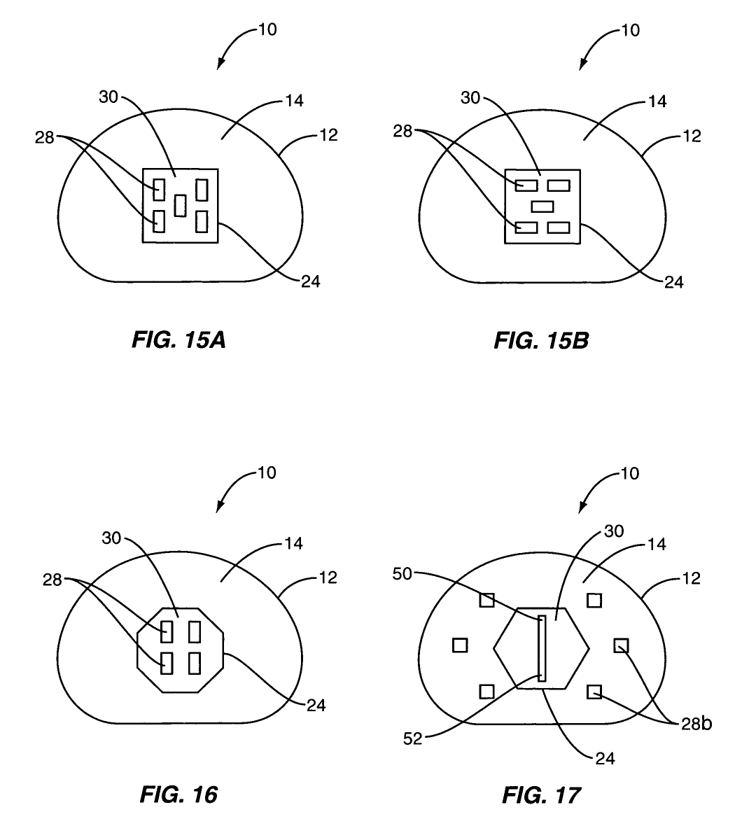

The opening 24 may have a geometric shape to allow the cover 30 to be positioned at a variety of different angular positions. FIGS. 15A and 15B illustrate an embodiment with the opening 24 and the cover 30 each being rectangular. The cover 30 further includes teeth 28 that are oriented in one direction. Cover 30 may be attached in a first direction as illustrated in FIG. 15A with the teeth 28 oriented in the first direction. In this orientation, the implant 10 may be inserted into the intervertebral space 91 using a first approach direction. Cover 30 may also be oriented in other directions, such as illustrated in FIG. 15B. In this orientation, the same implant 10 may be inserted using a second approach angle. The geometry of the opening 24 and cover 30 dictate the possible angular options for different approaches. A rectangular geometry provides for four different approaches. FIG. 16 illustrates another embodiment with the opening 24 and cover 30 each having an octagon shape. This geometry provides for eight different approach directions each being 45° apart.

A variety of attachment mechanisms 32 may be utilized to attach the cover 30 to the body 12. In one embodiment, the cover 30 attaches within the opening 24 with an interference fit. As illustrated in FIG. 2A and 5, legs 39 extend outward from an underside of the plate 31. The legs 39 are elastically deformable to fit within the opening 24 during insertion, and spring outward and expand beyond the size of the opening 24. The outer edges of the legs 39 contact the underside of the plate 31 and prevent removal. FIG. 6 illustrates another embodiment with the plate 31 including a threaded edge 33 that engages threads on edge of the opening 24. Another embodiment features pins on the underside of the cover 30 that engage with apertures in the body 12. One skilled in the art will understand that a variety of different attachment means may be used to attach the cover 30 to the body 12.

In some embodiment as illustrated in FIGS. 5 and 6, cover 30 does not include teeth 28. In other embodiments, the cover 30 includes teeth 28. Teeth 28 on the cover 30 may be the same size and shape as those on the body 12, or may be different. Further, cover 30 may include a first type of teeth 28 with a first shape and size, and a second type of teeth with a second shape and size.

FIG. 7 illustrates another embodiment with the opening 24 including notches 41 and a lip 42. The notches 41 extend into the superior surface 14 and include a lower support 45 recessed below the level of the surface 14. The lip 42 is in communication with the notches 41 and is positioned on the underside of the superior surface 12. Cover 30 includes extensions 34 that extend outward from the plate 31 and are sized to fit within the notches 41. During attachment, the cover 30 is placed over the opening 24 and the extensions 34 are placed within the notches 41 with an underside of the extensions contacting the lower supports 45. Rotation of the cover 30 causes the extensions 34 to move away from the notches 41 and under the lips 42 to prevent removal. In one embodiment, the notches 41 include a protuberance that moves beyond a rib in the notch 41 to maintain the position of the cover 30 in the closed orientation. A groove 35 may be positioned within the surface of the plate 31 to receive a tool for rotating the cover 30 during attachment and detachment.

In the embodiment of FIG. 7, the position of the notches 41 provide for positioning the cover 30 at a variety of different rotational positions. Each extension 34 is able to fit within each notch 41. With this embodiment including four separate extensions 34 and eight notches 41, the cover 30 can be positioned at increments of about 45°. Various numbers of notches 41 may be positioned about the opening 24 to provide a variety of angular positions for the cover 30. In the embodiment of FIG. 7, each of the teeth 28 are oriented in a particular direction. Therefore, the cover 30 can be positioned within the opening 24 to align the teeth 28 for a particular approach direction. By way of example, the cover 30 may be orientated at a first angular position for a first approach direction, such as a posterior approach. The cover 30 may be orientated at a second angular positioned for a second approach direction, such as a lateral approach.

In one embodiment as illustrated in FIG. 7, the body 12 may not include teeth 28. Therefore, the teeth 28 positioned on the cover 30 engage the vertebral member 90 and prevent expulsion. In another embodiment as illustrated in FIG. 2B, the teeth 28 on the cover 30 are shaped and sized like the teeth 28 on the body 12. Each of the teeth 28 includes a slanted orientation, and the teeth 28 on the cover 30 are oriented in the same direction when the cover 30 is attached to the body 12. FIG. 8 illustrates another embodiment with the teeth 28a on the cover 30 including a slanted orientation. Teeth 28b on the body 12 are substantially symmetrical and non-directional. The symmetrical teeth 28b may be inserted from a variety of approach angles, and the teeth 28b on the cover 30 are positioned to facilitate the specific insertion approach.

Cover 30 may also include a keel 50 as illustrated in FIG. 12. Keel 50 extends outward from the plate 31 a distance beyond the teeth 28. Keel 50 is substantially straight and facilitates insertion of the implant 10 into the intervertebral space 91. Keel 50 may be substantially perpendicular to the plate 31, or may be positioned at an angle. The keel 50 may include serrations or teeth 51 on the edges 52 to facilitate insertion into the intervertebral space 91 and prevent expulsion. Further, a leading edge of the keel 50 may be tapered to facilitate insertion.

Keel 50 may further be positioned on an embodiment with a geometric opening 24 and cover 30 that provides for selective angular positioning. FIG. 17 illustrates an embodiment with the opening 24 and cover 30 each having six sides. The keel 50 is positioned on cover 30 and can be angularly positioned at 60° angular intervals. The cover 30 may not include teeth 28, or may include symmetrical teeth 28b either of which provides for positioning from various angular directions.

The implant 10 may include a single opening 24 positioned on the superior 14 or inferior 16 surfaces that lead into the interior section 20. In another embodiment, more than one opening 24 is positioned on one of the superior or inferior surfaces 14, 16 that lead into the interior section 20. The openings 24 may be the same or different shapes and sizes. Further, implant 10 may include one or more openings 24 on each of the superior 14 and inferior 16 surfaces. FIG. 13 illustrates an embodiment including an annular body 12 with a first opening 24a on the superior face 14 and a second opening 24b through the inferior face 16. Each opening 24a, 24b leads into the interior section 20. In one use, the body 12 may be used without covers 30a, 30b as an annular spacer.

A first cover 30a is sized to extend across the first opening 24a, and a second cover 30b is sized to extend across the second opening 24b. In another use, one of the covers 30a, 30b may be placed across the respective opening and bone growth material may be placed within the interior section 20. The implant 20 may be inserted into the vertebral space 91 with the uncovered opening facilitating fusion. Alternatively, both covers 30a, 30b may be attached to the body 12 prior to insertion into the vertebral space 91.

The implant 10 may be constructed from biocompatible metal alloys such as titanium, cobalt-chrome, and stainless steel. The implant 10 may be constructed from non-metallic materials, including for example, ceramics, resins, or polymers, such as UHMWPE and implantable grade polyetheretherketone (PEEK) or other similar materials (e.g., PAEK, PEKK, and PEK). The implant 10 may be constructed of synthetic or natural bone or bone composites. The body 12 and the cover 30 may be constructed of the same or different materials. In one embodiment, body 12 is constructed of a non-resorbable material and the cover or covers 30a, 30b are constructed of a resorbable material. A predetermined time after insertion, the cover or covers 30a, 30b are resorbed to expose the interior section 20.

The embodiment of FIGS. 2A and 2B include a substantially straight posterior sidewall 18_{P}. In another embodiment as illustrated in FIG. 4, implant 10 includes a curved sidewall 18_{P} with the body 12 including an overall curved or kidney shape. Particularly, the posterior side P of implant 10 may be arcuate such that the posterior sidewall 18_{P} curves inwardly towards the anterior side A of implant 10.

A variety of materials may be positioned within the interior section 20 to facilitate fusion of the vertebral members. Suitable examples of bone growth promoting substances include bone morphogenic protein (BMP), LIM mineralization protein (LMP), demineralized bone matrix (DBM), mesenchymal stem cells, blood platelet gel, and biological materials. Other materials are disclosed in U.S. Patent Application Publication Nos. 2005/0203206 and 2006/0025861, each herein incorporated by reference.

FIG. 3A illustrates the body 12 being tapered on both the superior surface 14 and anterior surface 16. Another embodiment illustrated in FIG. 14 includes a tapered surface on the superior surface 14 with the inferior surface 16 being substantially flat. In other embodiments, only the inferior surface 16 includes a tapered surface 22 and the superior surface 14 extends in a direction substantially perpendicular to the opening 24. Regardless of the embodiment, the tapered surface 22 distracts the adjacent vertebral members 90 to facilitate insertion of the implant 10 into the intervertebral space 91.

One embodiment includes accessing the spine S from an anterior approach. Other applications contemplate other approaches, including posterior, postero-lateral, antero-lateral, oblique, and lateral approaches to the spine S. The implant 10 may be used in various regions of the spine S, including the cervical, thoracic, lumbar and sacral regions.

In the embodiments illustrated, the opening or openings 24 are positioned on the superior and/or inferior surfaces 14, 16. In other embodiments, the opening or openings 24 may extend into the sidewall 18. Further, a single opening 24 may be sized and positioned to extend from both superior and inferior surfaces 14, 16, and the sidewall 18. Likewise, a cover 30 may extend over the various surfaces to extend across the opening 24.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An intervertebral implant to fit within an intervertebral space formed between vertebral members, the implant comprising:
a body including inferior and superior surfaces and a sidewall that forms an interior section;
an opening extending through one of the inferior and superior surfaces and into the interior section;
a cover sized to fit across the opening and prevent escape of bone growth material within the interior section; and
a plurality of teeth positioned on the cover, the teeth extending outward above the body to engage one of the vertebral members.

2. The implant of claim 1, further comprising a plurality of apertures extending through the inferior and superior surfaces, the apertures being smaller than the opening.

3. The implant of claim 1, wherein the cover includes a plate sized and shaped to substantially match the opening and an attachment mechanism to attach to the body and position the cover across the opening.

4. The implant of claim 3, further comprising a plurality of apertures extending through the plate, the apertures being smaller than the opening.

5. The implant of claim 1, wherein the cover further comprises a keel that extends outward beyond the plurality of teeth.

6. The implant of claim 1, wherein each of the plurality of teeth are oriented in a common direction to facilitate insertion of the implant into the intervertebral space and resist expulsion.

7. An intervertebral implant to fit within an intervertebral space formed between vertebral members, the implant comprising:
a body including inferior and superior surfaces and a sidewall that forms an interior section;
an opening extending through one of the inferior and superior surfaces and into the interior section;
a cover including a plate and an attachment mechanism that attaches to an edge of the opening to position the plate across the opening; and
a plurality of teeth positioned on the cover and orientated in a common direction, the teeth extending outward above the body when the cover is attached to the body to engage one of the vertebral members;
the cover being rotatably positioned within the opening to align the plurality of teeth in a desired direction to facilitate insertion into the intervertebral space.

8. The implant of claim 7, wherein the cover and the opening both have a circular shape.

9. The implant of claim 7, wherein each of the teeth include a first angled surface and a second surface that extends substantially perpendicular from the body, the first angled surface being longer than the second surface.

10. The implant of claim 7, further comprising a plurality of second teeth positioned on the cover, the plurality of second teeth including a different shape and size than the plurality of teeth.

11. The implant of claim 7, wherein the cover further comprises a keel that extends outward from the plate beyond the plurality of teeth.

12. The implant of claim 7, further comprising a second opening extending through one of the inferior and superior surfaces and into the interior section, and a second cover sized to fit across the second opening.

13. The implant of claim 12, wherein the opening and the second opening are each positioned on one of the inferior and superior surfaces.

14. The implant of claim 7, wherein the body includes an annular shape with a second opening extending through one of the inferior and superior surfaces.

15. The implant of claim 7, wherein the opening further includes notches that extending into the body and the cover further includes extensions sized to fit within the notches.

16. The implant of claim 15, wherein the body further includes lips positioned adjacent to the notches to capture the extensions when the cover is attached to the body.

17. An intervertebral implant to fit within an intervertebral space formed between vertebral members, the implant comprising:
a body including first and second surfaces and a sidewall that forms an interior section;
an opening extending through the first surface and into the interior section;
a cover including a plate and an attachment mechanism that attaches to an edge of the opening to position the plate across the opening, the plate being substantially equal in size to the opening; and
a plurality of teeth positioned on the cover and orientated in a common direction, the teeth extending outward above the body when the cover is attached to the body to engage one of the vertebral members;
the cover being selectively positionable in a variety of angular orientations to orient the plurality of teeth to insert the implant in a variety of different approach directions.

18. The implant of claim 17, wherein the first surface of the body is substantially toothless.

19. The implant of claim 17, wherein the opening has a polygonal shape.

20. An intervertebral implant to fit within an intervertebral space formed between vertebral members, the implant comprising:
a body including first and second surfaces and a sidewall that forms an interior section;
an opening extending through the first surface and into the interior section;
a cover including a plate and an attachment mechanism that attaches to an edge of the opening to position the plate across the opening; and
a keel that extends outward from the cover to engage one of the vertebral members;
the cover being selectively positionable in a variety of angular orientations to orient the keel to insert the implant into the intervertebral space in a variety of different approach directions.

21. The implant of claim 20, further comprising a plurality of teeth positioned along an edge of the keel to contact the vertebral member.

22. The implant of claim 20, wherein the plate and the opening are substantially equal in size.

23. An intervertebral implant to fit within an intervertebral space formed between vertebral members, the implant comprising:
an annular body including inferior and superior surfaces and an outer sidewall with an open central section; and
a first cover sized to extend across the open central section at the inferior surface and a second cover sized to extend across the open central section at the superior surface, each of the covers including a plate sized to fit across the central section and an attachment mechanism that attaches to an edge of the body.

24. The implant of claim 23, further comprising a plurality of teeth positioned on each of the first and second covers.

25. The implant of claim 24, wherein the plurality of teeth on each of the first and second covers are orientated in a common direction to resist expulsion.

26. The implant of claim 24, wherein the inferior and superior surfaces are substantially smooth.

27. The implant of claim 23, wherein the first and second covers and the central section are circular.

28. The implant of claim 23, further comprising notches extending into the body at the first and second openings, and the first and second covers each include extensions sized to fit within the notches to attach the covers to the body.

29. The implant of claim 24, wherein the inferior and superior surfaces further include a plurality of second teeth.

30. A method of positioning an implant within the intervertebral space formed between vertebral members, the method comprising the steps of:
placing bone growth material through an opening within one of the interior and superior faces and into an interior section of a body;
attaching a cover across the opening to prevent the bone growth material from escaping;
adjusting the cover relative to body and aligning oriented teeth on the cover in a predetermined direction; and
inserting the implant with the attached cover in the predetermined direction and into the intervertebral space with the teeth contact the vertebral member.

31. The method of claim 30, wherein the step of adjusting the cover relative to the body comprises the cover and the opening being circular and rotating the cover within the opening.

32. The method of claim 30, wherein the step of adjusting the cover relative to the body comprises inserting extensions on the cover into notches with the body and rotating the cover with the extensions moving underneath lips on the body.

33. The method of claim 30, further comprising adjusting the cover relative to body and aligning the oriented teeth on the cover in a second predetermined direction.

34. The method of claim 33, wherein the opening has a polygonal shape and the step of adjusting the cover relative to the body comprises aligning corresponding edges on the cover and the opening.
